(19) United States
(12) Patent Application Publication
(10) Pub. No.: US 2006/0204400 A1
(43) Pub. Date: Sep. 14, 2006

(54) Process for separation of dispersions and an apparatus

(76) Inventors: Christoph Blattert, Schallstadt (DE); Andreas Schoth, Merdingen (DE); Reinhold Jurischka, Neuenburg (DE); Isam Tahhan, Stegen (DE); Wolfgang Menz, Dettenheim (DE)

Correspondence Address: RONALD E. GREIGG;GREIGG & GREIGG P.L.L.C., 1423 POWHATAN STREET, UNIT ONE, ALEXANDRIA, VA, 22314, US

(21) Appl. No.: 11/285,200

(22) Filed: Nov. 23, 2005

Related U.S. Application Data
(60) Provisional application No. 60/630,196, filed on Nov. 24, 2004.

(51) Int. Cl. G01N 33/48

(52) U.S. Cl. 422/68.1

(57) ABSTRACT

A process for separation of dispersions or suspensions by applying an external pressure gradient between a feed reservoir and at least one waste reservoir in such a way that the dispersion flows into a microchannel system. At least one fraction is separated through an opening and via at least one target channel. Different volume flows in a waste channel and a target channel of the microchannel system are set by the selection of an external pressure gradient. The various phases in a dispersion or suspension are separated and concentrated further by a series arrangement of structures of bend arcs. An apparatus for carrying out the process connects the feed reservoir and at least one waste reservoir via a feed channel, at least one bend arc and further channels, respectively, the fractions of the dispersion or the suspension separated substantially within the at least one bend arc.

### CROSS REFERENCE TO RELATED APPLICATIONS

This application is based on U.S. Provisional Application No. 60/630,196 filed Nov. 24, 2004.

### BACKGROUND OF THE INVENTION

1. Field of the Invention

The invention relates to an improved process for separation of dispersions and to an apparatus for carrying out this process.

2. Description of the Prior Art

The separation of dispersions for chemical, physical and/or biological analysis of substances plays a major role in analytical laboratory technique. The separation of dispersions is of major importance for many applications. In the biological area the separation of suspensions gets more and more important.

Already known processes for separation of small amounts of a suspension such as those which are required in environmental and bioanalysis are time consuming and require a large amount of apparatus. Spinning the sample is often used for the separation of particles and solvents. Separation into phases of different density is in this case carried out by means of the centrifugal force which acts on the sample during the acceleration. Centrifuges are relatively large and expensive apparatuses and the sample capacity is limited.

A further known process for separation of particles and solvents is filtration. The degree of separation is governed by the size of the filter pores with small pores resulting in an increase in the flow resistance, and quickly becoming blocked. The system-dependent dead volume is also relatively large so that the filtration of small sample amounts is associated with a comparatively high loss.

In the case of the separating nozzle disclosed in patent specification DE 32 038 42, the individual fractions are separated by circulation in a curved gap. The traditional application of this process is nuclear technology and in the removal of dusts from gases. However, this process has the disadvantage that the gap ends which form the nozzle, result in a transverse flow, which in turn leads to flow disturbances and as a consequence of this, comparatively large amounts of energy are required which may cause undesirable side effects for biological materials.

WO 03/033096 is related to a method and separating module for the separation of particles from a dispersion in particular of blood corpuscles from blood. A separating module, suitable for carrying out this method comprises a substrate with flow channels including a feed channel for the supply of the dispersion to a branching of a first discharge channel for leading fluid with reduced particle concentration away from the branching and a second drainage channel for leading fluid with increased particle concentration away from the branching. The fluid flows into the second drainage channel so much faster than into the first drainage channel that the particles at the branching preferably flow into the second drainage channel as a result of the differing flow speeds.

WO 03/031015 A1 discloses a method of and an apparatus for the separation of suspensions in which an external pressure gradient is applied between an inlet reservoir and outlet reservoirs so that the suspension flows into a microchannel system. At least one fraction is drawn off directly after an elbow bend through a suction opening leading to a suction channel. The adjustment of various volumetric flows in the supply and drain channels of the microchannel system is achieved by means of selecting the external pressure gradient. The various phases of a suspension are further separated and concentrated by means of a series of elbow bends. The device for carrying out the method connects an inlet reservoir in at least two outlet reservoirs by means of an inlet run, an elbow bend and two channels arranged at an angle of β≦90°.

From the publication “Separation of Blood Cells and Plasma in Microchannel Bend Structures” by C. Blattert, R. Jurischka, A. Schoth, P. Kerth, W. Menz, SPIE Optics East 2004, Philadelphia/Pa., USA, it is known that biological applications of micro assay devices require easily implementable on-chip microfluidics for separation of plasma or serum from blood. This is achieved by a new blood separation technique based on a microchannel bend structure developed within a collaborative biochip project. Different prototype polymer chips have been manufactured with UV-LIGA process and hot embossing technology. The separation mechanisms have been identified and the separation efficiency of these chips has been determined by experimental measurements using human blood samples. Results show different separation efficiencies for cells and plasma up to 100% depending on microchannel geometry, hematocrit and feed velocity. This technique leads to an alternative blood separation method as compared to existing microseparation technologies.

From the publication “Separation of Blood Samples and Plasma in Microchannel Bend Structures” (see above) separation chip geometries are known which are different concerning the width of the channels and the bend radius. In table 1 of this publication the varying bend radii in mm and the length of the channels are given in greater detail.

From the publication “Separation of Blood in Microchannel Bends”, C. Blattert, R. Jurischka, I. Tahhan, A. Schoth, P. Kerth, W. Menz in Proceedings of the 26^{th} Annual International Conference of the IEEE EMBS, San Francisco, Calif., USA, Sep. 1-5, 2004, it is known that most clinical chemistry tests are performed on cell-free serum or plasma. Therefore, micro assay devices for blood tests require integrated on-chip microfluidics for separation of plasma or serum from blood. This is achieved by a new blood separation technique based on a microchannel bend structure. Different prototype polymer chips have been manufactured with an UV-LIGA process and hot embossing technology. The separation efficiency of these chips has been determined with samples of human whole blood as well as diluted blood samples. The results show different separation efficiencies up to 90% for blood cells and plasma depending on microchannel geometry as well as cell concentration. As compared to the present microfluidic devices for the separation of blood cells like filters or filtration by diffusion the microchannel bend is an integrated on-chip blood separation method which combines the advantages of rapid separation times and simple geometry.

### SUMMARY AND ADVANTAGES OF THE INVENTION

An object of the present invention is to provide a simple microfluidic process and an apparatus which allows separation of solids from liquids and the separation of the various phases in a dispersion or a suspension.

A further object of the present invention is to provide for a structure designed such that it can be integrated in an “on the chip” installation system.

According to the invention, these objects are achieved by the following features:
a) an external pressure gradient is applied between at least one feed reservoir, at least one waste reservoir and at least one target reservoir such that the dispersion flows into at least one curved microchannel,
b) at least one fraction of dispersion or suspension being separated through at least one opening and via at least one target channel by application of centrifugal force and plasma skimming, and
c) said at least one fraction being separated within the at least one curved microchannel after having passed at least about ⅓ of the length of said curved microchannel in the direction of flow.

For the separation, two mechanisms are responsible. The first mechanism is the centrifugal force in the bend region of the curved microchannel, which causes different settling velocities based on density differences between particles and the surrounding fluid of the dispersion. In laminar capillary flow which is typical for microfluidic systems, particles show an axial migration, as a consequence of which the particle enriched core of the flow is deflected to the outer wall of the bend of the curved microchannel and a fluid enriched layer is obtained at the inner wall of the curved microchannel. Preferably, within this area of the curved microchannels, at least one target channel branches off, to convey the fluid enriched layer towards at least one target reservoir.

The second separation mechanism is the plasma skimming effect. If the flow rates in diverging bifurcations are significantly different, particles tend to enter into the branch with the higher flow rate. The pressure forces, due to different flow velocities on the upper and lower side of the particle and the shear stress on the particle both point to the branch having the higher flow rate. As a consequence thereof, the particles tend to follow this respective branch. According to the present invention, the separation of a dispersion or of a suspension, for example in biological applications, is achieved by application of centrifugal force in combination with plasma skimming. The centrifugal force is created by a curved microchannel through which the dispersion or suspension flows. The flow velocity on the dispersion or suspension is imposed by an external pressure gradient, applied to the system and resulting in a flow velocity profile of the dispersion or suspension. Preferably the curved microchannels comprise a funnel-shapes widening in direction of flow of the dispersion or suspension, increasing the separation efficiency. The funnel-shaped widening within the curved microchannel provides a larger area on the respective inner section of the curved microchannel in which a phase of lower density flows which is to be separated from a phase with higher concentration on the outer section of the curved microchannel. The funnel-shaped widening of the curved microchannel, i.e. the increasing cross-section thereof in the direction of flow, provides for a larger area within which a plasma-phase is flowing whereas in the outer area of the curved microchannel a particle enriched phase is flowing. Particularly in connection with a plurality of target channels assigned to the inner section of the curved microchannel, the funnel-shaped widening of the curved microchannel significantly increases separation efficiency.

In combination with the centrifugal force, plasma skimming is achieved by different flow rates in the at least one waste channel and the at least one target channel. The flow rate generated in the at least one waste channel exceeds the flow rate in said at least one target channel. This can be achieved by variation of the pressure gradients between the at least one feed reservoir and the at least one waste reservoir or a variation of pressure gradients between the at least one feed reservoir and the at least one target reservoir. Further, different flow rates can be achieved by variation of the external pressure gradient between the at least one feed reservoir, the at least one waste reservoir and the at least one target reservoir. Further, plasma skimming can be achieved by different geometries of the channels. For example, the cross-section of the target channel is smaller than the cross-section of the at least one waste channel. Further, different flow rates can be generated by different lengths of the channels, the length of the at least one target channel exceeding the length of the at least one waste channel. Furthermore, upon layout of the microfluidic system, flow resistance within the at least one waste channel is low, whereas a flow resistance within the at least one target channel is high, thus creating a high flow rate within the at least one waste channel and a lower flow rate in the at least one target channel.

According to the present invention a combination of the application of centrifugal force and plasma skimming results in a high separation efficiency in connection with a funnel-shaped widening of the curved microchannel to which the opening into the at least one target channel is provided on the respective inner side thereof, i.e. in that area where the phase of the dispersion to be separated, i.e. plasma is flowing. The phase of the dispersion having a higher density, i.e. a particle phase of the dispersion is concentrated in the outer area of the curved microchannel.

The curved microchannel preferably is designed as a bend arc. An arrangement of bend arc structures in series allows the phase with the lower density to be concentrated in such a way that, after a first separation of the liquid phase, the target channel becomes the feed channel for a subsequent bend arc, with the enriched phase being successively separated further via the subsequent bend arc.

The principle of arranging bend arc structures in series is also suitable for further separation and concentration of the various phases in a dispersion. The fractions in the waste reservoir may be passed to an analysis process or to other processes not given in greater detail herein below.

The apparatus for carrying out the process described above, including its variants is characterized according to the invention by at least one feed reservoir, at least one waste reservoir, and at least one target reservoir which are connected via a feed channel comprising a bend arc and two channels forming a bifurcation located within the bend arc, arranged at an angle β of ≦90°. The bend arc has an angle α of ≧45°. The larger the angle α the longer the centrifugal force acts on the dispersion thus increasing separation efficiency significantly.

The bend arc may run in all three spatial directions. For geometric reasons, with the channel arrangement in two dimensions, the arc angle with a constant arc radius is <360°. Arc angles of more than 360° can then be achieved only by a spiral channel arrangement with an arc radius which becomes constantly smaller. However, an arrangement such as this has the disadvantage that it occupies a large amount of lateral space. The advantage of the use of the third dimension is the capability to achieve arc angles of more than 360° with a constant arc radius. One major advantage of an arrangement such as this is that target channels can easily additionally be fitted to a helical channel structure.

N waste reservoirs are connected by (N-1) bend arcs. The bend arcs may be produced from metal, glass, silicon, ceramics, or a natural or synthetic polymer. The apparatus is integrated in a microfluidic analysis system for analysis of the various fractions in the dispersion.

The process according to the invention and the apparatus have the advantage over the prior art that the very small dimensions of the channels allow the dispersion to be separated onto the microchip, and that only relatively small substance volumes in the range from picoliters to microliters are required.

In comparison to the process described in the prior art, the system according to the present invention is controllable and can be designed independently of the amount of liquid. The system according to the invention thus ensures reproducibility. In comparison to the process described in the prior art, the system according to the present invention furthermore achieves better separation efficiency independently of the fluidic characteristics (only density differences are significant in the system according to the invention).

Typical fluctuations in dispersion composition and dispersion fluidics can thus be compensated for via pressure gradients in the system according to the invention which is not possible with the system described in the prior art. Since, furthermore, the geometry of the system described in the prior art is governed by liquid, owing to the capillary force, and only very small separated amounts are available, the system according to the present invention can be used more universally and thus for different applications. Suspensions can be separated in the same way.

According to further aspects of the present invention the microfluidic device comprises at least one feed reservoir, at least one waste reservoir, at least one target reservoir, connected by a bend arc, a feed channel, a waste channel and a target channel, the waste channel and target channel forming a bifurcation following the bend arc. The orientation of the target channel forming a bifurcation with the waste channel within the bend is chosen such that an opening is provided within the bend arc, seen in the fluid direction of the fluid to be processed. Further, the ratio of channel depth to channel width (aspect ratio) is chosen within 1 and 10 for the at least one target channel. According to a preferred geometry, the feed channel is chosen to have a 60 μm width and 60 μm depth. The waste channel is chosen to be of a width of 90 μm and a respective depth of 60 μm whereas target channel is of the width of 20 μm and has a depth of 60 μm, each of said channels having a length of 3 mm, respectively.

The use of the microfluidic device is not limited for application on suspensions but can also be used to provide for a separation of dispersions such as gas/solid mixtures.

A further advantage according to the present invention is given by reinforcing structures such as cross bars and by local broadenings of the channels, particularly in the target channels for increase of stability which significantly enhances manufacturing reliability and a reproduction of the device upon manufacturing thereof. The reinforcing structures may have the shape of ribs provided in a manufacturing tool, to give an example. By means of the reinforcing structures such as cross bars or local broadenings of the channel, the manufacturing of a microfluidic device can be improved significantly, since upon removal of the manufacturing tool from a substrate, the microstructures are prone to collapse. Within the reinforcing structures the stability of the microfluidic device according to the present invention is improved significantly.

### BRIEF DESCRIPTION OF THE DRAWINGS

The present invention will be described hereinafter by reference to the accompanying drawings, in which:

FIG. 1 shows a first exemplary embodiment of the apparatus according to the present invention for separation of a phase having a first density from a dispersion,

FIG. 2 shows a second exemplary embodiment of the apparatus according to the present invention, in which the series separation of a phase having a first density results in concentration of a phase having a second density,

FIG. 3 shows a third exemplary embodiment of the apparatus according to the invention in which the series separation of the phases of different density is achieved,

FIG. 4 shows the apparatus according to the invention integrated in a microchip laboratory,

FIG. 5 shows an embodiment of the present invention having a plurality of target channels in substantially parallel configuration,

FIG. 6 shows a first embodiment of reinforcing structures, resulting in transverse connections between two parallelly extending target channels,

FIG. 7 shows a second embodiment of reinforcing structures, applied to the target channels,

FIG. 8 shows a cross section through the substrate of the microfluidic device being covered on top thereof by a cover element, and

FIG. 9 shows a target channel having an aspect ratio, which is different from the aspect ratio as given in the embodiment according to FIG. 8.

### DESCRIPTION OF THE PREFERRED EMBODIMENTS

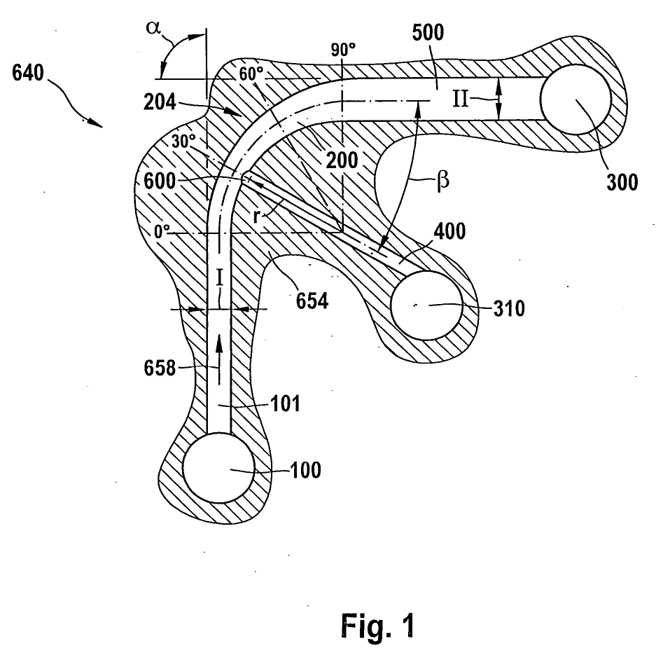

In FIG. 1 a first exemplary embodiment of the apparatus according to the invention for separation of a phase having a first density from a dispersion is given. For the following, it is noted that within a microfluidic device according to the present invention, separation of phases of dispersions or suspensions is effected between a phase having a first density from a phase having a second density. The first density exceeds the second density. For biological applications such as separation of blood, the phase having a high, first density is the cellular enriched phase whereas the phase having the second, lower density is the plasma phase which is to be separated.

The structure according to the invention comprises a feed reservoir 100, a feed channel 101, at least one waste reservoir 300, a target reservoir 310, a target channel 400, a waste channel 500 and a bend arc 200. The bend arc 200, which may run three-dimensionally in all three spatial planes, is defined by the arc angle α and the arc radius r.

The microfluidic structure is filled with a dispersion or a suspension via the feed reservoir 100. A pressure gradient is applied between the feed reservoir 100, the waste reservoir 300 and the target reservoir 310, respectively, such that fractions of the dispersion or the suspension enter the feed channel 101. After passing through the feed channel 101, a parabolic velocity distribution is achieved within the feed channel 101 from the initially uniform velocity distribution. On the one hand, the wall friction force (which is proportional to the pressure gradient applied to the feed channel) in this case acts on the particles dissolved in the dispersion. This leads to the formation of a laminar flow within the feed channel 101. An edge flow is decelerated and the core flow is accelerated owing to the continuity equation; the laminar flow profile is formed completely. One feature of laminar flow condition is the parabolic velocity distribution over the cross section of the feed channel 101. The largest velocity occurs in the centre and the lowest velocity in the edge areas. The shear rates behave in precisely the opposite way.

In principle, two mechanisms create the separation of the dispersion. The first mechanism to be mentioned is the centrifugal force in the bend region of the bend arc 200 which causes different settling velocities based on density differences between particles and the fluid surrounding the particles. In laminar capillary flow which is typical for microfluidic systems, particles show an axial migration as the consequence of which the particle enriched core of the stream of the dispersion is deflected to the outer wall of the arc bend 200 and a fluid enriched layer of the dispersion is obtained at the inner wall of the bend arc 200.

The second separation mechanism is the plasma skimming effect. If the flow rates in diverging bifurcations such as between the waste channel 500 and the target channel 400, are significantly different from one another, particles tend to enter the branch with the higher flow rate. The pressure forces, due to different flow velocities on the upper and lower side of the particle and the shear stress on the particle both point to the branch, i.e. the respective channel with the higher flow rate. As a result of this the particle will follow this respective branch, i.e. in this application the waste channel 500. According to the combination of the centrifugal forces effect and the skimming effect mentioned above, the opening 600 is located within the bend arc 200. The combination of the application of centrifugal force and plasma skimming enhance significantly, the separation efficiency of the microfluidic structure according to the present invention. Whereas the centrifugal force is created by the curvature of the bend arcs 200, 201, 202 and is increased by a funnel-shaped widening, i.e. an increase of cross-section of the bend arcs 200, 201, 202 in the flow direction, the plasma skimming effect is increased by establishing different flow rates within the at least one waste channel 500 and the at least one target channel 400. Different flow rates within said channels 400, 500, respectively, are achieved by variation of the external pressure gradients between the at least on feed reservoir 100 and the at least one waste reservoir 300 and/or between the at least one feed reservoir 100 and the at least one target reservoir 310. Concerning the geometry of the at least one target channel 400 and the at least one waste channel 500, a respective cross-section of the at least one waste-channel 500 is chosen in that way that the cross-section of the at least one waste channel 500 exceeds the cross-section of the at least one target channel 400. Furthermore, the geometry of the channels 400, 500 can be varied such that a length of the at least one target channel 400 exceeds the length of the at least one waste channel thus creating a higher flow rate in the at least one waste channel 500 as compared to the flow rate within the at least one target channel 400. Furthermore, the flow resistance within the at least one waste channel 500 is smaller as compared to the respective flow rate within the at least one target channel 400, thus creating a higher flow rate within the at least one waste channel 500 as compared to the flow rate within the at least one target channel 400.

The phase of the 1^{st} density, or the particles in the dispersion, are preferably gathered in the regions where the velocities are high and the shear rates thus are low. For the bend arc 200 this means that the flow conditions result in the phase of 1^{st} density being separated in the flow after ⅓ of the length of the bend arc 200 in the distal area thereof, while in contrast the phase of 2^{nd} density is concentrated in the proximal area thereof. The phase of 2^{nd} density may be removed directly within the bend arc 200 through an opening 600 and via a target channel 400. According to the illustration given in FIG. 1 said opening 600 is located substantially within the first bend arc 200. Said opening 600 for the at least one target channel 400 may be located advantageously after about ⅓ of the length of the bend arc 200 to allow for removal of the fluid enriched layer (phase having 2^{nd} density) at the inner wall of the bend arc 200 into the target channel 400. Preferably, the opening 600 is located between the 30° position but prior to the 90° position with respect to the radius of the bend arc 200. Thus, a preferred orientation of the opening 600, connecting the target channel 400 to the bend arc 200 is preferably chosen between about ⅓ of the length of the bend arc 200 in the flow direction of the dispersion but prior to the end of the bend arc 200, i.e. its exit into the subsequently arranged waste channel 500. In connection with FIG. 1, it is worthwhile mentioning that the cross section of the bend arc 200 widens in the flow direction as seen in FIG. 1 depicted by the funnel shaped area indicated by reference numeral 204.

The flow of the dispersion or a suspension in the microchannel system 640 is produced by means of a physical potential. The physical potential may be an electrical, thermal or hydraulic potential. The electrical potential is produced by application of different electrical voltages to the feed channel 101 and the respective waste channels. In particular, this allows electrically conductive dispersions to be moved through said microchannel system 640. A thermal potential is produced by heating or cooling areas of the feed channel 101 and the waste channel 500, see FIG. 5. This leads to a change in density in the dispersion and, as a result of the expansion or a result of shrinkage, to a movement of the dispersion within the microchannel system 640. The hydraulic potential difference which is required for movement of the dispersion is produced by application of a pressure medium, preferably a suitable gas medium such an inert gas (N₂) or noble gas (helium).

The choice of the respective medium for production of a potential or of a force for movement of the dispersion through the arc bend 200 and channels 101, 400, 500 in a microchip which may be connected is in this case governed in particular by the physical characteristics which are inherent in the dispersions and the respective components thereof.

The proposed structure according to the present invention allows for simple forming of the separating effects without any moving or active fluidic components such as pumps or other pressure sources or valves for controlling the fluid flow. The separating effects mentioned above are the creation of the centrifugal force in the bend region of the arc bend 200 which causes different settling velocities based on density differences between particles and the surrounding fluid. In laminar capillary flow which is typical for microfluidic systems particles show an axial migration as a consequence of which a particle enriched core of the stream is deflected to the outer wall of the arc bend 200 and the fluid enriched layer is obtained at the inner wall 200 where said opening 600 to the target channel 400 is located. The second separation effect is the plasma skimming effect. If the flow rates in diverging bifurcations are significantly different, particles tend to enter the branch of the bifurcation with a higher flow rate. Pressure forces due to different flow velocities on the upper and lower side of the particle and the shear stress on the particle both point to the branch with the higher flow rate. As a result of that the particle tends to follow the branch having the higher flow rate.

Furthermore, the bend arc 200 can be manufactured at low costs and with a variable geometry using a large number of materials as are used in microsystem technology, for example natural and synthetic polymers, metals, glass, quartz, silicon or ceramics. Without wishing to imply any restriction, etching and milling, in particular as well as injection-molding or die-casting methods may be used as a suitable production method; outstanding results have been achieved by manufacturing microfluidic devices by means of X-ray-LIGA-methods or by UV-LIGA-methods. The LIGA-manufacturing method allows for obtaining high quality microfluidic devices with respect to the precision achievable. Further, it has been proven advantageously to combine the milling technology with electrochemical milling and to combine the milling technology with laser-structuring. Upon application of the UV-LIGA-method, an aspect ratio of about 10 is achievable, the aspect ratio being defined as the ratio of channel depth to channel width which will be described in greater detail below.

Within the bend arc 200, the feed channel 101 branches into two channels, the target channel 400 and the waste channel 500, respectively. The target channel 400 is connected to the bend arc 200 on the proximal side with respect to the bend arc 200 by means of an opening 600, located within the bend arc 200. The target channel 400 is set up at a specific angle β, and opens into the waste reservoir 310. The waste channel 500, however, opens into the waste reservoir 300. The respective widths of the target channel 400 and the waste channel 500 may in this case be the same as that of the feed channel 101 or may even be smaller. Furthermore, said target channel 400 and the waste channel 500 do not necessarily need to have the same diameter. The precise dimensions are in this case governed by the fractions to be separated and by the proportions of the fractions in the initial dispersion. The dispersion may be separated into two or more phases by the flow conditions in the bend arc 200 after passing through the bend arc 200. The phase with the lower density is passed through the opening 600 and through the target channel 400 to the target reservoir 310 from which it can easily be removed.

The remaining component of the dispersion flows via the waste channel 500 into said waste reservoir 300.

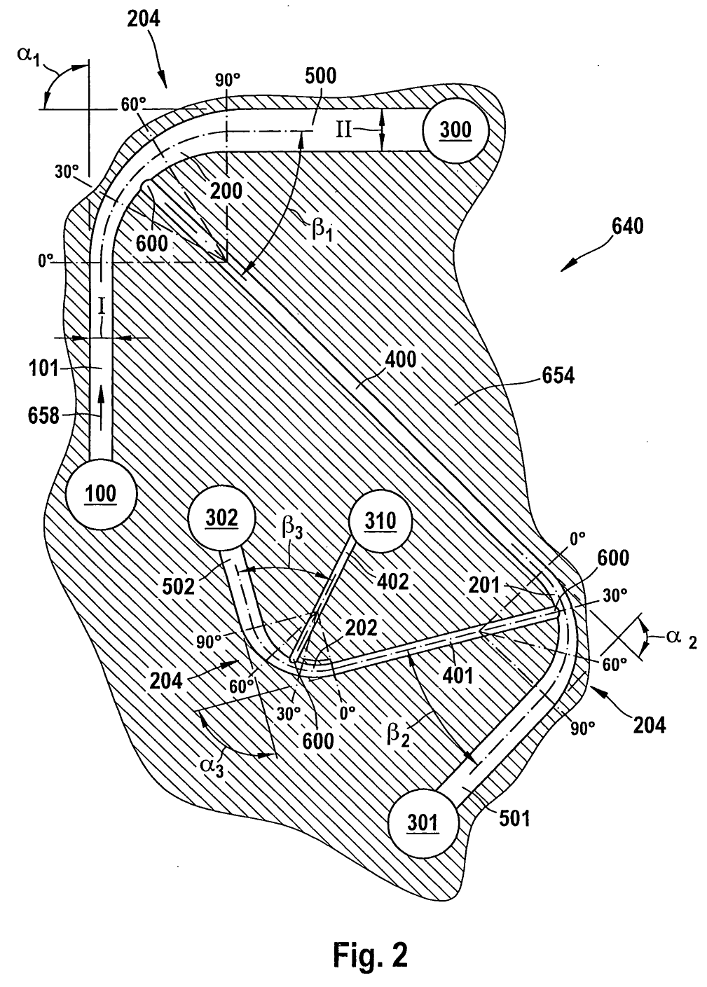

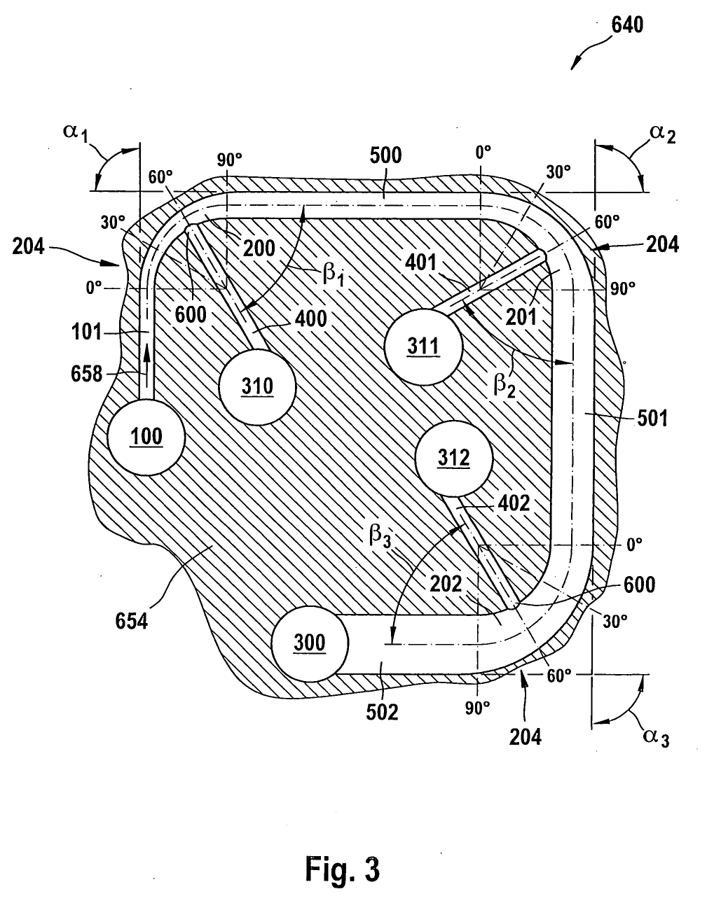

Series arrangements of bend arc structures 200, 201, 202 are described in connection with FIGS. 2, 3, respectively, and are used to concentrate a phase or to separate the dispersion into two or more phases.

The angles α₁, α₂, α₃, of the bend arcs 200, 201, 202, respectively, the angle β of the target channels 400, 401, 402 and the length of the feed channels 101 may in this case be the same or may differ from one another. The diameter of the channels may likewise remain constant or may differ in particular may be smaller. In particular, the target channel 400 may be of a smaller width as compared to the width of the waste channel 500. It is to be noted, that analogous to the embodiment given in FIG. 1, the bend arc structures 200, 201, 202 each are shaped with a funnel-like widening 204, within which in the flow direction, a cross section of said bend arcs 200, 201, 202 continuously widens up, from cross-section I to cross-section II.

The arrangement shown in FIG. 2 represents an example of a preferred embodiment of the present invention for the concentration of the phase of low density.

The dispersion is introduced into the microfluidic system 640 from the feed reservoir 100. A first separation step of the liquid phase takes place in the bend arc 200. The target channel 400 now becomes a feed channel for the next subsequent bend arc 201. The enriched phase is separated further successively via the bend arcs 201 and a further bend arc 202 and, finally, arrives at the 3^{rd} waste reservoir 302 having a higher concentration than the initial concentration in the dispersion. The fractions in the first, second and third waste reservoirs 300, 301 and 302, respectively, may be passed in precisely the same way as the fractions gathered in the target reservoir 310 to an analysis process or other processes not described herein in detail.

By way of example, FIG. 3 shows a further preferred arrangement of the microchannel system 640 which is used to separate different phases of a highly complex multiple phase dispersion.

The phase with the lowest density is separated after said 1^{st} bend arc 200 and can be extracted from said other 1^{st} target reservoir 310. Further, phases with rising density can be separated successively via said subsequently arranged bend arcs 201, 202, respectively, and are extractable via said further target reservoirs 311, 312, respectively.

In principle, further cascading of the structures of bend arcs 200, 201, 202, respectively, as well as a combination of the arrangement mentioned in the two exemplary embodiments given in FIG. 2, 3, respectively, that have been described herein above, are conceivable.

The geometric shape of the apparatus, that is to say the length, width, depth and the cross-sectional shape of the microchannels, the arc radii r_{1-N}, the arc angle α_{1-N} and the angle β_{1-N} of the waste channels 400, 401, 402 as well as the position of the further target reservoirs 310, 311, 312, respectively, and the positions of the further waste reservoirs 300, 301, 302, respectively, depend on the physical characteristics of the media to be separated.

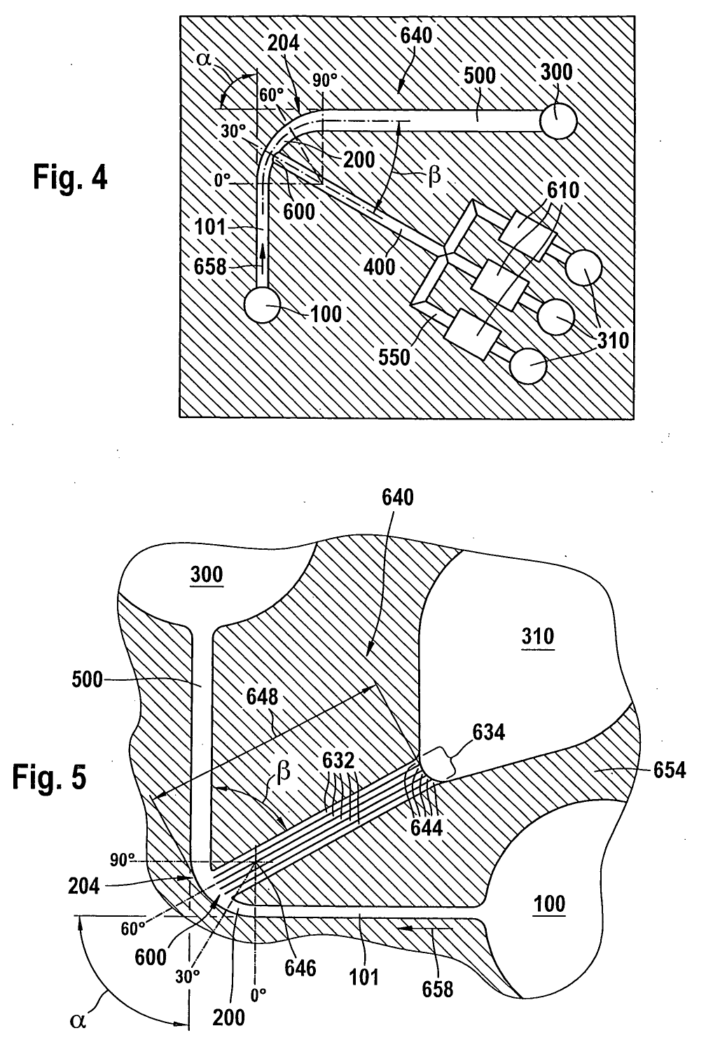

The apparatus according to the present invention may be integrated into a microchip laboratory by way of example as described in the German patent application 199 49 551 A1 or U.S. Pat. No. 5,858,195. This is schematically illustrated in FIG. 4. One or more of the phases separated by the bend arc 200 may be subjected to the same or different analysis processes, for example physical, chemical, toxicological, pharmacological or biochemical/biological analysis. This allows complex substance mixtures to be analyzed, for example biological liquids such as blood, urine or lymphs, surface water or seepage water. By means of the microfluidic device having a microchannel system 640 and being applied on a substrate, a separation of particles out of gases is conceivable as well.

According to the illustration given in FIG. 5 a further advantageous embodiment of the present invention comprises a plurality of plasma channels, extending substantially in parallel to one another.

According to FIG. 5 a feed reservoir 100 is provided on a substrate 654. On the substrate 654 furthermore a first waste reservoir 300 is arranged. The feed reservoir 100 and the first waste reservoir 300 are connected via a feed channel 101, a first bend arc 200 and via a waste channel 500. The direction of flow of the dispersion is indicated by reference numeral 658 of the dispersion from the at least one feed reservoir 100 to the at least one waste reservoir 300.

Within the first bend arc 200 at the opening 600 a plurality of target channels 632 branch off from the first bend arc 200. The plurality of target channels 632 establishes a microchannel system 640, in which each of the single target channels extend in a parallel configuration 634 with respect to one another. The single target channels of the plurality of target channels comprise an opening 600, which is substantially located within the first bend arc 200. The plurality of target channels further comprises a common opening 644. The openings 600 and 644, respectively, connect the first bend arc 200 with a target reservoir 310. Each of the target channels out of the plurality of target channels 632, extending substantially in parallel configuration 634 with respect to one another is separated from each other by separating walls 642 having a wall thickness exceeding a width 650 of each of the single respective target channels out of the plurality of target channels.

The target channels out of the plurality of target channels 632 preferably have a length 648 between 0.5 and 10 mm, and more preferably between about 2 and 8 mm and most preferably about 3 mm. All of the channels 101, 632, 500 according to the embodiment given in FIG. 5 preferably have a length of 3 mm.

With respect to the geometry of the microchannel system 640 being provided on the substrate 654 each of the channels, i.e. each of the plasma channels out of the plurality of plasma channels 632, has an aspect ratio of channel depth 652 to channel width 650 which is advantageously chosen between 1 and 10. The aspect ratio between depth 652 of the respective channel and width 650 of the respective channels to one another may vary between 1 and 10 and is equal for all of the target channels out of the plurality of target channels. Further, it can be derived from the embodiment given in FIG. 5 that the feed reservoir 100 provides a fluid flow by applying a pressure gradient to the microfluidic system 640 in the flow direction 658. Thus, the fluid, i.e. the dispersion, is driven from the feed reservoir 100 to the respective first waste reservoir, labelled with reference numeral 300.

The separation efficiency achievable with the embodiments of FIGS. 1 to 5, respectively, according to the present invention can be determined by the following equation:

$$Separation efficiency = 1 - \frac{particle concentration within target reservoir 310}{particle concentration within feed reservoir 100} .$$

The separation efficiency is a function of the flow rates within the target channel 400 or within the target channels out of the plurality of target channels 632 to the flow rate in the waste channel 500. The separation efficiency is determined by the skimming effect, mentioned in detail above and by the application of a centrifugal force which is a function of the flow velocity and the widening 204 of the respective bend arc 200. In the embodiment given in FIG. 5 a widening 204 of the bend arc 200 in flow direction 658 is given in a smaller scale as compared to the widening 204 of the respective arc bend 200, 201, 202, respectively according to the embodiments given in FIGS. 1 to 4, respectively.

In a preferred embodiment of the apparatus according to the present invention, the feed channel 101 has a width 650 of about 60 μm and a depth 652 of about 60 μm, whereas the waste channel 500 according to the embodiment given in FIG. 5 has a width 650 of about 90 μm and a depth 652 of about 60 μm. Furthermore, it is added that each of the target channels out of the plurality of target channels 632 has a channel width 650 of about 20 μm and a channel depth 652 of about 60 μm. In a preferred embodiment of the apparatus according to the present invention the length 648 of the channels, preferably is about 3 mm. For the preferred embodiment given in FIG. 5 the same applies concerning the location of the openings 600 within the bend arc 200. Seen in the flow direction 658 of the dispersion from the feed reservoir 100 to the waste reservoir 300 the opening 600 is located within said arc bend 200 in such a way that the opening 600 is located at the inner wall of the bend arc 200. Seen in flow direction 658, the opening 600 is preferably located at an angle of between 30° and 90°, i.e. substantially within the second half of the bend arc 200. The location of the opening 600 at which either the plurality of target channel branches off from the bend arc 200 or a single target channel 400 branches off to a target reservoir 310 enhances the performance of the low density phase of the dispersion to enter said plurality of target channels connected to the target reservoir 310. Reference numeral 646 depicts the center of the radius of the bend arc 200. In the embodiment given in FIG. 5 the 0° position and the 90° position are shown, covering the entire angle α of the bend arc 200. The angle β depicts the orientation of the plurality of target channels 632 with respect to the orientation of the waste channel 500, connecting the bend arc 200 with the waste reservoir 300.

In the illustration according to FIG. 5 the plurality of target channels comprises six target channels 632, being separated from one another by the separation walls 642. The respective wall thickness of the separation walls 642 is labeled with reference numeral 656 exceeding the width of the respective target-channels out of the plurality of target channels 632.

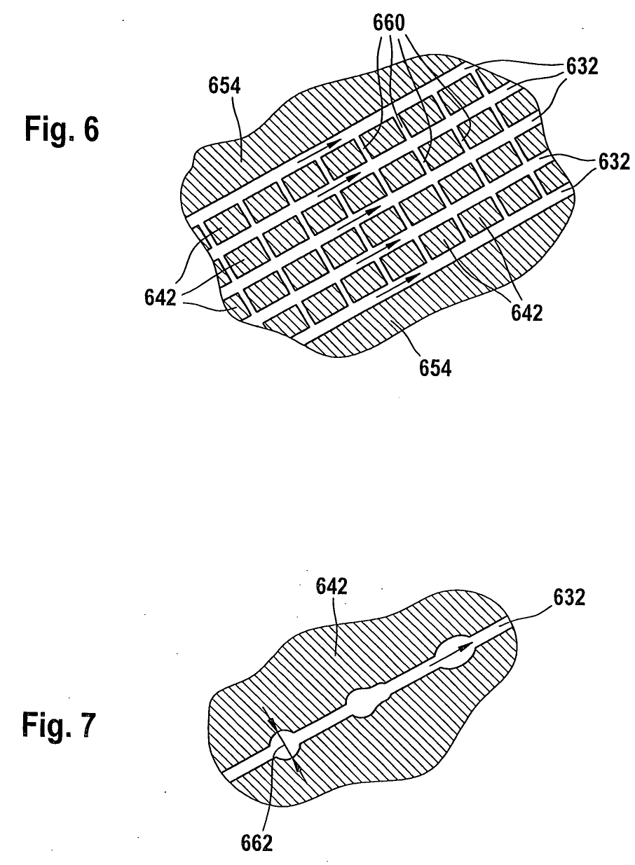

FIG. 6 shows a first embodiment of reinforcing structures provided on a microfluidic device. According to the illustration given in FIG. 6 two out of the plurality of target channels are shown in a larger scale. The target channels are separated from each other by separation walls 642. The separation walls 642 include gaps 660 which are formed by the tools for manufacturing the microfluidic device according to the present invention. The tool comprises cross bars which upon manufacturing of said microfluidic device form said gaps 660, each interconnecting said target channels connecting the microbend 200 via opening 600 to the target reservoir 310. The gaps 660 provided within the separating wall 642 given in a larger scale as well prevent the target channels out of the plurality of target channels 632 from collapsing upon removal of the manufacturing tool. The flow direction of the low density phase within the target channels shown in FIG. 6 is indicated by the arrows. It is conceivable to provide local broadenings 662 within the microchannel system 640 as show in greater detail in FIG. 7.

FIG. 7 shows a second embodiment of a reinforcing structure of microchannels out of a plurality of microchannels 632, the local broadenings indicated by reference numeral 662. The local broadenings 662 are preferably formed with the continuous wall structure, i.e. having no sharp edges or the like. Therefore, the local broadenings 662 are preferably manufactured as drop-shaped local broadenings or circular or oval broadenings applied to the channel and located adjacent to one another. The flow direction of the low density phase is shown by the arrow; the target channel is manufactured within the substrate 654 of the microfluidic device either being glass, metal, silicon, ceramics, natural or synthetic polymer. Depending on the method of manufacturing of the microfluidic device according to the present invention, other shapes of the local broadenings 662 are conceivable. The broadenings 662 allow for an easier removal of a manufacturing tool upon manufacturing of the plurality of target channels 632 in a substantially parallel configuration indicated by reference numeral 634.

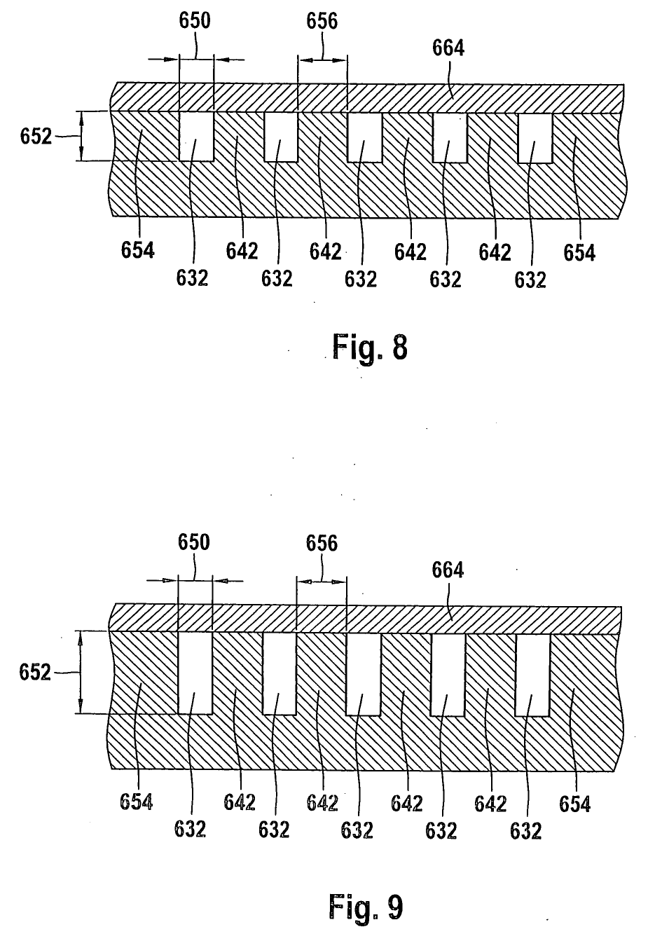

FIG. 8 shows a cross-section of a microfluidic device according to FIGS. 1 to 5, on a larger scale.

FIG. 8 shows a cross-section in the area of the plurality of the target channels 632. The single target channels are separated from one another by separation walls manufactured, i.e. milled or etched or structured by laser into said substrate 654 and are covered by a cover element 664. Although not shown in the embodiments given according to the FIGS. 1 to 5 described herein above, the microfluidic devices according to FIGS. 1 to 5 are covered by a cover element 664 comparable to the cover element 664 given in the cross-section according to FIG. 8.

In the embodiment according to FIG. 8 the plurality of target channels 632 comprises six single target channels, each being separated by separating walls 642. The respective width of a single target channel is depicted by reference numeral 650, the respective depth thereof is depicted by reference numeral 652. The aspect ratio is defined as the ratio between channel depth 652 to channel width 650. In the embodiment given in FIG. 8, the aspect ratio for each of the target channels out of the plurality 632 of target channels is about 2, whereas in the embodiment given in FIG. 9 the aspect ratio of the single target channel shown there is about 3. The value of the aspect ratio between channel depth and channel width depends on the method of manufacturing of the microfluidic device. A more reliable manufacturing of microfluidic device according to the present invention is achieved if the wall thickness of the respective separation wall 642, separating the target channels of the plurality of target channels 632 from one another exceeds the width 650 of said target channels, being arranged substantially in parallel configuration labeled 634 according to FIG. 5. The substrate 654 comprises the separating walls 642, since the respective target channels out of the plurality of target channels 632 are etched or milled into the substrate 654, whereas the cover element 664 schematically shown in FIG. 8 constitutes a separate element to close the microfluidic device according to the present invention.

The funnel-shaped widening 204 shown in the embodiments given in FIGS. 1 to 5 has a first cross-section I at which the dispersion or suspension, onto which an external pressure gradient is imposed, enters said bend arcs 200, 201, 202, respectively. The cross-section at the end (90°-position) is labeled with II. Further, it is worthwhile mentioning that the aspect ratio, i.e. the ratio between channel depth 652 and channel width 650 varied between 1 and 10. However, the aspect ratio of channel depth 652 to channel width 650 may adopt values between 3 and 20, depending on the manufacturing process and depending on the application, within which the microfluidic structure according to the present invention is used. Further it is worthwhile mentioning, that the funnel-shaped widening 204, in which the curved microchannels, i.e. the bend arcs 200, 201, 202, respectively, are designed allows for a significant improvement of separation efficiency when used in connection with a plurality 632 of target channels.

The foregoing relates to preferred exemplary embodiments of the invention, it being understood that other variants and embodiments thereof are possible within the spirit and scope of the invention, the latter being defined by the appended claims.

We claim:

1. A process for separation of dispersions or suspensions, in which spilt fractions are supplied via a microchannel system to one or more analysis areas, the process comprising,
the steps of applying an external pressure gradient between at least one feed reservoir, at least one waste reservoir and at least one target reservoir such that the dispersion or suspension flows into at least one curved microchannel, and
separating at least one fraction of dispersion or suspension through at least one opening and via at least one target channel by centrifugal force and by plasma skimming,
said at least one fraction being separated within said at least one curved microchannel after having passed at least ⅓ of the length of said curved microchannel in the flow direction of said dispersion.

2. The process as claimed in claim 1, wherein the centrifugal force is generated by the curvature of said curved microchannel.

3. The process as claimed in claim 1, wherein the centrifugal force is generated by the application of said external pressure gradient, determining the flow velocity of said dispersion or said suspension.

4. The process as claimed in claim 2, wherein the separation effect of the centrifugal force within said curved microchannels is increased by a widening of said curved microchannels in direction of flow.

5. The process as claimed in claim 1, wherein the plasma skimming effect is generated by a higher flow rate within at least one waste channel as compared to a flow rate in at least one target channel.

6. The process as claimed in claim 5, wherein said different flow rates within said at least one waste channel and in said at least one target channel are generated by variation of said external pressure gradients between said at least one feed reservoir and said at least one waste reservoir.

7. The process as claimed in claim 5, wherein said different flow rates in at least one waste channel and in at least one target channel are generated by variation of said external pressure gradients between said at least one feed reservoir and said at least one target reservoir.

8. The process as claimed in claim 5, wherein said different flow rates in said at least one waste channel and said at least one target channel are generated by variation of said external pressure gradient between said at least one feed reservoir, said at least one waste reservoir and said at least one target reservoir.

9. The process as claimed in claim 5, wherein said different flow rates in said at least one waste channel and said at least one target channel are generated by a lower flow resistance in said at least one waste channel as compared to the flow resistance within said at least one target channel.

10. The process as claimed in claim 5, wherein said different flow rates in said at least one waste channel and said at least one target channel by the selection of a cross-section of said channels the cross-section being selected such that said cross-section of said at least one waste channel exceeds said cross-section of said at least one target channel and a length of said channels is selected such that a length of said at least one target channel exceeds the length of said at least one waste channel.

11. The process as claimed in claim 1, wherein volume flows are set in said feed, waste and target channels of said microchannel system by the selection of said external pressure gradient.

12. The process as claimed in claim 1, wherein the flow of the suspension in the microchannel system is produced by means of a physical potential.

13. The process as claimed in claim 12, wherein the physical potential is a hydraulic potential, or an electrical or a thermal potential.

14. The process as claimed in claim 1, wherein a concentration of a phase with a lower density is separated by means of a series arrangement of bend arc structures such that after a first separation step of phases, at least one target channel forms a feed channel for a subsequently arranged bend arc an enriched phase being successively separated further via said at least one bend arc.

15. The process as claimed in claim 14, wherein the various phases in a dispersion are separated and concentrated further by a series arrangement of structures of bend arcs.

16. Apparatus having a microchannel system for carrying out the process as claimed in claim 1, the apparatus comprises at least one feed reservoir, at least one outlet reservoir and at least one target reservoir are connected via an feed channel, at least one bend arc and at least two further channels, said at least one bend arc having at least one opening for target channels out of a plurality of target channels, said opening being located within said at least one bend arc after ⅓ of the length of said bend arc in flow direction of said dispersion.

17. Apparatus according to claim 16, wherein said at least one bend arc comprises a funnel-shaped widening in flow direction of said dispersion or said suspension.

18. Apparatus according to claim 16, wherein at said opening of a plurality of target channels branches-off from said at least one arc bend, said plurality of target channels being located in substantial parallel configuration with respect to one another.

19. Apparatus according to claim 16, wherein said target channels out of said plurality of target channels each comprise gaps formed by a manufacturing tool having cross-bar sections assigned thereto to increase stability of a substantially parallel configuration of the plurality of target channels.

20. Apparatus according to claim 16, further comprising separating walls separating said target channels out of the plurality of target channels from one another, the separating wall thickness exceeding said channel width of said channels.

21. Apparatus according to claim 16, wherein said target channels out of the plurality of target channels comprise circular, oval or drop-shaped local broadenings for stabilization of said single target channels out of the plurality of target channels.

22. Apparatus according to claim 16, wherein said at least one arc bend is manufactured from metal, glass, silicon, ceramics or natural or synthetic polymers.

23. Apparatus according to claim 16, wherein said feed channel has a channel width of about 60 μm and a channel depth of about 60 μm, said waste channel has a channel width of about 90 μm and a channel depth of about 60 μm and each of said target channels out of said plurality of target channels has a channel width of about 20 μm and a channel depth of about 60 μm, wherein the number of target channels of the plurality or target channels is 6, and a length of said feed channel, said waste channel, and each target channel out of the plurality of target channels is about 3 mm.

24. Apparatus according to claim 16, wherein a channel length of each of said channels is chosen in the range between about 2 mm and 4 mm.

25. Apparatus according to claim 16, wherein said target channels out of said plurality of target channels, comprise an aspect ratio of channel depth to channel width between 1 and 10.

26. Apparatus according to claim 16, wherein said apparatus is integrated into a microfluid analysis system, or an analytical microsystem for analysis of various fractions in said dispersion or said suspension.

27. Apparatus according to claim 16, wherein an arc angle α, α₁, α₂, α₃ of said at least one bend arc is in the range of ≧45° and wherein n waste reservoirs are connected by means of bend arcs.
